(19) United States
(12) Patent Application Publication
(10) Pub. No.: US 2017/0136135 A1
(43) Pub. Date: May 18, 2017

(54) METHOD AND SYSTEM FOR DECONTAMINATING CAPS OR NECKS OF CONTAINERS BY PULSED ELECTRON BOMBARDMENT

(71) Applicant: SIDEL PARTICIPATIONS, Octeville Sur Mer (FR)

(72) Inventor: Guy FEUILLOLEY, Octeville Sur Mer (FR)

(73) Assignee: SIDEL PARTICIPATIONS, Octeville Sur Mer (FR)

(21) Appl. No.: 15/317,291

(22) PCT Filed: May 19, 2015
(86) PCT No.: PCT/FR2015/051296

§ 371 Date: Dec. 8, 2016

(30) Foreign Application Priority Data
Jun. 11, 2014 (FR) 14 55305

(51) Int. Cl. A61L 2/08; B65B 55/04; B67B 3/00

(52) U.S. Cl. CPC A61L 2/087; B67B 3/003; B65B 55/04; A61L 2202/23

(57) ABSTRACT

A method for decontaminating caps (2) or necks of containers by electron bombardment, the method including: an operation of the passage or positioning of the caps (2) or necks of containers in front of an electron bombardment window (8), the opening of the caps (2) or necks of the containers facing the window (8); and an operation of electron bombardment of the caps (2) or necks of the containers, during the passage or positioning of the caps or necks of the containers in front of the window (8); the bombardment being carried out by way of a pulsed electric field including a series of electrical pulses of determined frequency, duration and intensity.

The invention relates to the field of the sterilization of caps or necks of containers.

More particularly, the invention relates to a method and a system for decontaminating caps or necks of containers that make it possible to cover in an optimal manner all of the surfaces of these caps or necks.

Containers such as tubes, jars, flasks, cardboard food cartons or bottles made of PET (polyethylene terephthalate) are most often intended to contain common products of consumption, for example beverages, pharmaceutical products, or cosmetic products. Containers, such as bottles (in particular made of PET), are typically obtained via a stretch-blow-molding method starting from parisons, for example preforms or intermediate containers that have previously already undergone a first forming operation. The parisons as well as the caps of the containers are initially stored in a non-sterile environment.

The cardboard food cartons comprise a plugging device, consisting of a connected neck, closed by a plug. The manufacturing of a carton generally comprises a step of gluing the neck at the level of an opening located on one of the faces of the carton. Generally, these cartons, their necks, as well as the caps that are designed for them are also initially placed in a non-sterile environment. Consequently, before any filling and closing of the containers, the latter, their necks, as well as their caps should first undergo a method of decontamination in a sterilization chamber.

One known approach consists in spraying a sterilizing agent on the inside surfaces of the caps, necks and containers, for example hydrogen peroxide (H₂O₂), and in causing its evaporation by thermal action. Such an approach calls for spraying the agent over all of the surfaces of the containers, necks and caps; however, certain surfaces remain difficult to reach. Furthermore, the containers/necks/caps should be exposed to the agent for a predetermined time that is both long enough to ensure an effective sterilization, but also short enough so as to limit any damage by heating, running the risk of impairing these surfaces. Finally, such a method requires, if appropriate, a rinsing step so as to ensure that any trace of the product has been eliminated. Such an approach involves extended treatment times and turns out to be complex to implement.

Other known methods consist in carrying out the step of sterilizing containers via an accelerated electron bombardment on their surfaces. These methods make it possible to break the DNA bonds of any microorganism, or to create secondary particles that will then react with the microbial cells, thus leading to their elimination.

Contrary to the chemical route, these methods do not necessitate the rinsing step and do not leave any potential residual trace of chemical agent. In addition, the use of a low-energy electron beam (less than 1 MeV) makes it possible to limit the interactions with the material of the object that is to be decontaminated. By way of example, the document JPH06142165 proposes irradiating an object of complex shape, such as a cap, by a low-energy electron beam. Accelerated electrons form this electron beam, some of whose electrons collide with the gas molecules of the irradiated medium, thus creating dispersed electrons. After propagation, the electron beam, consisting of direct and dispersed electrons, then reaches the surfaces of the object and sterilizes them. The irradiated surfaces of the object furthermore induce reflected and/or secondary electrons that make it possible to sterilize the surfaces that are not directly irradiated.

However, the use of a low-energy beam involves a beam current (i.e., an anode current) of low value, most often on the order of about 10 mA. With these current values being low, the quantity of accelerated electrons turns out to be limited, just like their penetration into the material (several μm) and their back-scattering. So as to ensure the complete elimination of any microorganism, a minimal electron dose is to be produced. Consequently, so as to deposit a sufficient lethal dose of electrons on the surface of the object that is to be treated, generally on the order of about 10 kGy, a treatment time of several seconds is usually necessary. The treatment time of an irradiated object is a particularly critical parameter. Actually, an extended time of exposure of an object to electronic radiation runs the risk of creating undesirable effects on the object, namely discoloration, degradation, cross-linking phenomena, or else migration of odors. The approaches of the state of the art only manage to limit these problems partially, however.

One object of this invention is to eliminate all of the above-mentioned drawbacks.

Another object of this invention is to cover all of the surfaces of caps or necks of containers with complex shapes, having zones that cannot be covered directly by an incident electron beam.

Another object of this invention is to reduce the decontamination time of the caps or necks of containers with complex shapes, while improving the effectiveness of treatment, i.e., the bacteriological reduction rate, on the surfaces of these caps or necks of containers.

For this purpose, a method is proposed, according to a first aspect, for decontaminating plugs or necks of containers by electron bombardment, each cap comprising a roof, a body projecting from a peripheral edge of the roof, this body having an opening opposite the roof, ribs projecting from an inside face of the body and/or an inside face of the roof, each neck comprising ribs and an opening, the ribs having shadow zones, with this method comprising:
An operation for passage or positioning of the caps and/or necks of containers in front of an electron bombardment window, with the opening of the caps and/or necks of containers being turned toward this window;
An electron bombardment operation of caps and/or necks of containers, during the passage or positioning of the caps and/or necks of containers in front of the window;
   with the bombardment being carried out by means of a pulsed electrical field that comprises a series of electric pulses of predetermined frequency, duration and intensity in such a way as to obtain primary electrons and back-scattered electrons, respectively making possible the decontamination of exposed zones and shadow zones of the caps or necks.

Various additional characteristics can be provided, by themselves or in combination:
The frequency is encompassed in a range of between 50 and 500 Hertz;
The frequency of the electric pulses is 100 Hertz;
The duration of the electric pulses is encompassed in a range of between 5 and 250 nanoseconds;
The duration of the electric pulses is 10 nanoseconds;
The intensity of the electric pulses is between 1 and 20 kiloamperes;
The intensity of the electric pulses is 5 kiloamperes.

According to a second aspect, a system for decontaminating caps or necks of containers by electron bombardment is proposed, each cap comprising a roof, a body projecting from a peripheral edge of the roof, with this body having an opening opposite to the roof, ribs projecting from an inside face of the body and/or an inside face of the roof, each neck comprising ribs and an opening, with the ribs having shadow zones, this system comprising:
Means for passage or positioning of caps or necks of containers in front of an electron bombardment window, with the opening of the caps or necks of containers being turned toward this window;
Means for electron bombardment of caps or necks of containers, during the passage or positioning of the caps or necks of containers in front of the window, by means of a pulsed electric field comprising a series of electric pulses of predetermined frequency, duration and intensity in such a way as to obtain primary electrons and back-scattered electrons, respectively making possible the decontamination of exposed zones and shadow zones of caps or necks.

Advantageously, this system comprises a device for transport of caps that are adjacent to one another, along a transport path and at a predetermined speed.

Advantageously, in this system, the transport device is created by a set of rails.

Other objects and advantages of the invention will become evident from the description of embodiments, provided below with reference to the accompanying drawings in which

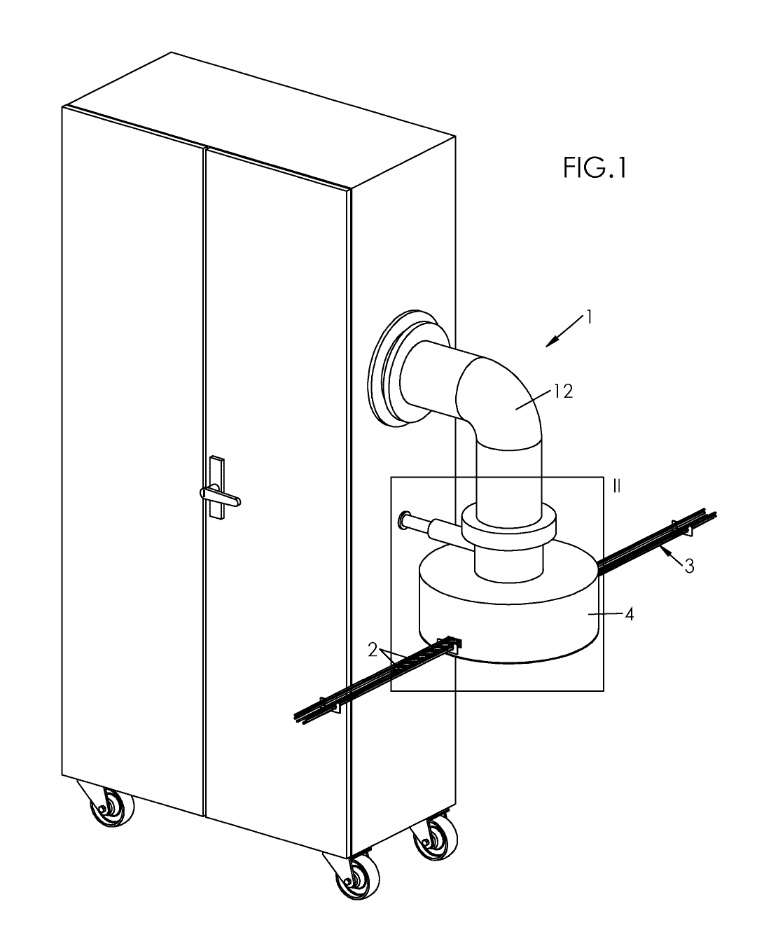

FIG. 1 illustrates a system that comprises an electron gun according to an embodiment;

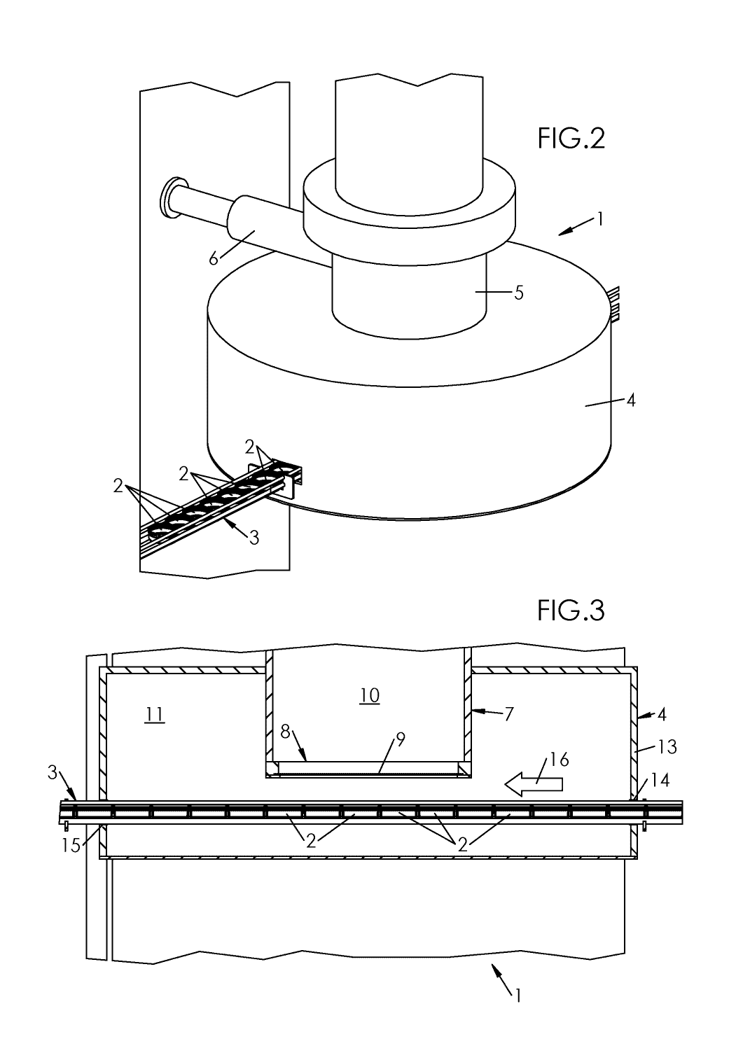

FIG. 2 illustrates an enlargement of a portion of the system that comprises the electron gun according to an embodiment;

FIG. 3 illustrates an enlarged cutaway view of the system that comprises the electron gun according to an embodiment;

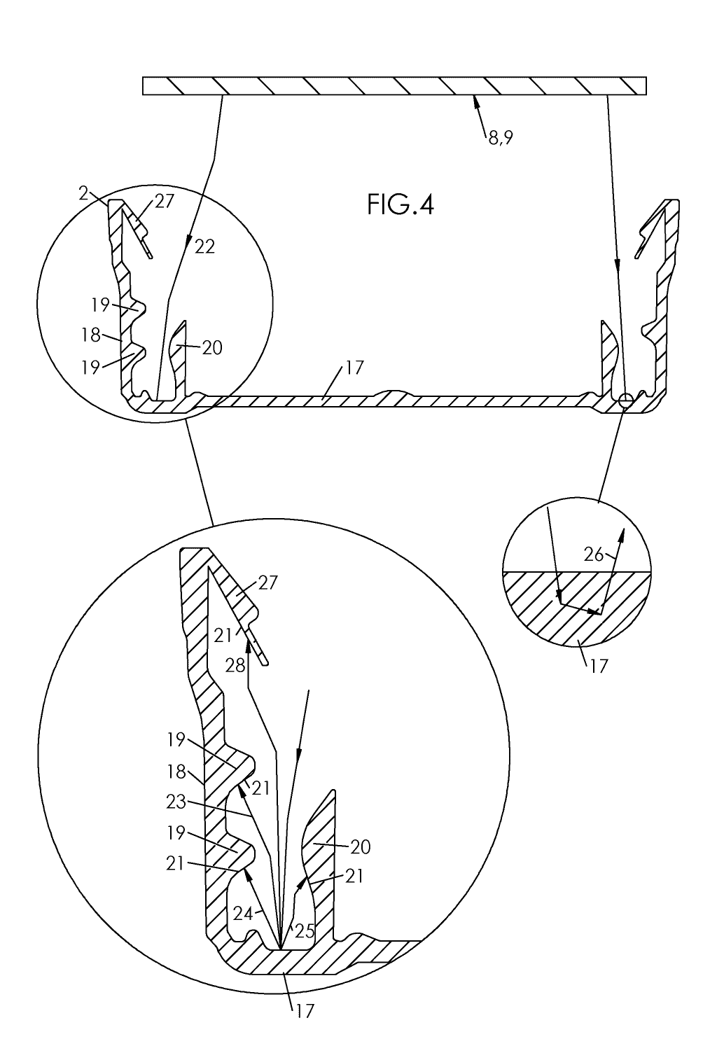

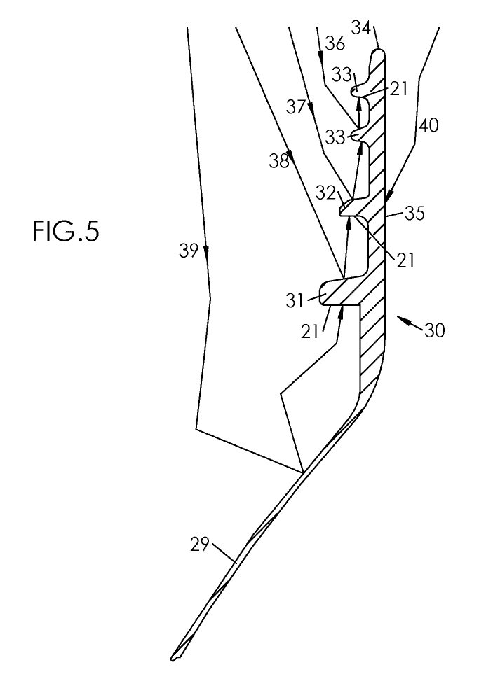

FIG. 4 illustrates a cutaway view of a container cap, as well as the various electron trajectories obtained from a pulsed electron beam;

FIG. 5 illustrates a cutaway view of a container neck, as well as the different electron trajectories obtained from a pulsed electron beam.

FIG. 1 shows a system 1 that comprises an electron gun, making it possible to generate a high-intensity electron flow. Advantageously, the generated electron flow at the exit of this gun is a pulsed electron flow/beam, used to bombard caps 2 and/or necks of containers for the purpose of their decontamination. Here, different embodiments that are applied to the caps 2 are described, but it is understood that these modes are all also applicable to the above-cited container necks. Using a transport device 3, these caps 2 pass into a sterilization chamber 4, i.e., a closed and sterile chamber that comprises the pulsed electron gun. Passage is defined here as a continuous temporal transport. According to another embodiment, the caps 2 are positioned in the sterilization chamber 3 in a sequential manner, i.e., step by step, for example via the transport device 3. The embodiment of all of these elements is described in detail below.

FIG. 2 is a detail on an enlarged scale of Zone II that is shown in FIG. 1. In this figure, the caps 2 of containers, the transport device 3, and the sterilization chamber 4 that are mentioned above are observed.

According to various embodiments, the electron flow/beam at the exit of the gun is formed by a set of electrons, with the latter being accelerated via the application of a potential difference between two electrons, respectively a cathode and an anode. The cathode is placed in a closed space 5, for example a “vacuum” chamber, i.e., at a pressure of very low value, for example less than 10⁻⁵ bar, ensured by a pumping device.

Advantageously, the creation of such a vacuum prevents the potential collision of electrons with gas molecules, then running the risk of creating a loss of energy for these electrons. The pumping device is connected to the space that is closed by means of a pipe 6. The anode constitutes one of the outside faces of the closed space under vacuum. The electron stream can be emitted, by way of example, in the direction of the anode by an explosive emission cathode, with this cathode and anode constituting a diode. By way of non-limiting examples, the explosive emission cathode that constitutes the diode can be made of graphite, stainless steel, copper, carbon or any other material that is known for the production of this type of electrode. Advantageously, this cathode does not comprise a filament.

In contrast to the filament diodes, the use of an explosive emission cathode diode has the following advantages:
Providing higher current densities and therefore larger electron doses for the decontamination of objects;
Emitting over a wide surface (example: 200 cm²), ensuring a more homogeneous distribution of electrons independently of the form of a filament;
Not requiring the installation of a heating device for the emission of electrons;
Not having a service life dependent on a filament (rupture of the filament), thus preventing any emission of electrons;
With no risk of short-circuiting internal to the diode, induced by a particle that is detached from material, extracted in particular from the filament, and temporarily interrupting the electronic emission.

FIG. 3 is a cutaway view of FIG. 2. In this figure, the caps 2 of containers, the transport device 3, the sterilization chamber 4, as well as the anode 7 ensuring both the closing, and therefore the isolation, of the vacuum space and the formation of an electron bombardment window 8 are observed.

The anode 7 is placed downstream in relation to the cathode in the direction of movement of the electrons and is made in the form of a unit of conductive metal, for example copper.

So as to allow the accelerated electrons to pass into the atmosphere, the former is pierced in its center and covered by a fine metal sheet 9, typically with a thickness on the order of several tens of μm, able, for example, to be made of titanium or aluminum. The thickness of the metal sheet 9 is selected in such a way as to make airtight the gap between the cathode and the anode 7, while allowing accelerated electrons coming from the cathode and impacting this sheet to pass through it.

The thus produced anode 7 constitutes an electron bombardment window 8 that makes possible the passage of accelerated electrons between the gap 10 of the closed space and an outside, for example gaseous, environment 11, such as ambient air. Advantageously, the way in which the conductive metal unit of the anode 7 is pierced conditions the shape of the electron beam that passes through the surface of the metal sheet 9 of the anode 7. Thus, the form of the electron beam and therefore the opening of the electron bombardment window 8 can be selected according to different geometries, by way of non-limiting examples in rectangular, circular or else annular shape.

By way of example, FIG. 3 illustrates an opening, and therefore a window 8, which is rectangular. In addition, so that the sheet 9 of the electron bombardment window 8 does not fail under the pressure difference between the gap 10 and the external environment 11 (relative to, for example, the outside atmospheric pressure):
According to one embodiment, a thickness of the sheet 9 and an opening of the window 8 that can ensure its rigidity, for example openings in the form of striae, are selected;
According to another embodiment, the surface of the anode 7 can be produced in a curved manner toward the inside of the closed space under vacuum 10.

In addition, it will be ensured, for the preceding reason, that the sheet 9 covering the anode 7 will be kept at a low enough temperature via the installation of suitable cooling means, not shown. The anode 7 can be designed, for example, in such a way as to comprise heat dissipation zones, or else be cooled by having a cooling fluid circulate along the latter through channels.

Advantageously, the electron beam that is obtained at the exit of the electron gun is homogeneous enough to cover all of the exposed surfaces of the object that is to be treated. By way of example, the surface of the electron bombardment window 8 is sized in such a way as to cover a surface that is considerably larger than the exposed surface of the bottom of a cap 2 that is centered in relation to this window 8.

The electron gun further comprises power-supply means, making it possible to establish a potential difference between the anode 7 and the cathode, so as to accelerate the electrons emitted by the cathode. The cathode is, for example, fed by an electrical energy source (not shown), while the anode 7 is grounded. According to various embodiments, so as to generate a pulsed electron flow at the exit of the electron gun, a continuous electrical energy source will be used, for example a high-voltage power supply coupled to means making it possible to store the electrical energy, for example a capacitive or inductive storage.

By way of example, a Tesla transformer coupled to a shaping line PFL (English acronym of “Pulse Forming Line”), or any other power-conditioning device, for example a Marx generator, is used. Advantageously, a switch makes it possible to control the pulse time (pulse) of the electrical energy of the beam, stored for a charging period of the electron gun. This switch is coupled to a conductor, placed in an insulation sheath. By way of example, in FIG. 1, the conductor in its insulation sheath is connected to the curved part 12 of the system 1. The conductor is connected to the cathode of the diode of the electron gun and ensures the junction between the cathode and the transformer, by means of the switch, thus feeding the diode by a pulsed voltage. A potential difference is thus created between the cathode and the anode 7, making possible the acceleration of the electrons emitted by the cathode into the gap 10.

A high-intensity pulsed electron flow is therefore obtained at the exit of the electron bombardment window 8. Advantageously, the use of a pulsed mode coupled with a low-energy electron beam (less than 1 MeV) makes it possible, in contrast to a continuous mode, to reduce the electrical insulation stresses of the electron gun and consequently to make it more compact. By way of example, effective electrical insulation of the transformer and the conductor is carried out via insulation by oil, and a thin steel or lead shield.

Advantageously, the pulsed electron beam that is obtained at the exit of the electron gun is used to bombard caps 2 of containers of complex shape, thus making possible their decontamination of any microorganism. Here, cap of complex shape is defined as any cap that comprises shadow zones, i.e., zones that cannot be reached directly by incident diffused electrons.

In the embodiments described below, the electrons obtained at the exit of the electron gun are diffused in air (external environment 11) and the caps 2 that are covered in this same environment. However, it is understood that any other gaseous or vacuum environment 11 can be used for the diffusion of electrons and the decontamination of caps 2.

According to various embodiments, caps 2 of complex shapes are brought into a sterilization chamber 4, in front of the electron bombardment window 8 of the electron gun, with the opening of the caps being turned toward this window 8. Sterilization chamber is defined as a hermetic and sterile closed space, comprising sterilization/decontamination means. For example, with reference to FIGS. 2 and 3, this chamber 4 is made using insulating metal surfaces 13 (example: lead/steel) that consist of a cylindrical volume whose axis of revolution is centered around the anode 7. This volume is pierced in such a way as to comprise an inlet opening 14 and an outlet opening 15 through which the device 3 for transport of caps 2 passes, thus making possible their channeling under the electron bombardment window 8 formed by the anode 7. The sterilization chamber 4 thus, in this embodiment, consists of the system 1 that comprises an electron gun. According to other embodiments, the sterilization chamber 4 is independent of the system 1 that comprises an electron gun and that comprises in its interior part or all of this system 1.

According to an embodiment that is illustrated in FIG. 3, the caps 2 pass laterally and in a single direction, parallel to and downstream from the electron bombardment window 8 of the anode 7. By way of example, the arrow 16 indicates a direction of lateral passage of the caps 2. In this figure, the caps 2 are adjacent to one another and pass along a predetermined transport path and at a predetermined speed, using a preestablished transport device 3, here a rail set over which the caps 2 slide. The caps 2 can pass along these rails under the effect of gravity or else using mechanical means (wheels, pushers) or pneumatic means (blow guns).

Advantageously, such a rail system makes it possible to ensure that the opening of the caps 2 of the containers is well turned toward the electron bombardment window 8 of the electron gun, during the passage of the caps 2 under the former. However, any other transport device 3 that makes it possible to ensure this arrangement of caps 2 could be used—by way of non-limiting example a pneumatic transport device. According to another embodiment, the caps 2 are positioned step by step under the electron bombardment window 8.

Advantageously, the caps 2 of containers that pass (or that are positioned) in front of the electric bombardment window 8 undergo an operation of bombardment by the pulsed electron beam that is generated at the exit of the electron gun. FIG. 4 illustrates a cutaway view of a circular cap 2 of the container, as well as different trajectories of electrons obtained from the pulsed electron beam at the exit of the electron bombardment window 8, with the trajectories of these electrons making possible the decontamination of specific zones of the cap 2. Furthermore, it is understood that the description of this type of cap 2 is provided here by way of example. Actually, the different embodiments that are described apply just as well to other types of caps with complex shapes, for example “sport”-type caps or else pin capsules.

A cap of complex shape, such as the one that is illustrated in this figure, typically comprises:
A flat bottom 17, also called a “roof,”
A threaded body 18 (threads inside and/or outside) starting from a peripheral edge of the roof 17, with this body 18 having an opening opposite the roof 17,
Ribs 19 that project from an inside face of the body 18, generally projecting parts to screw and/or to ratchet, provided for coming into contact with the outside of the neck of the container,
A skirt 27 that is part of a guarantee strip, placed on the inside face of the body 18,
Ribs 20 projecting from an inside face of the roof 17, typically an annular projection that supports a sealing lip.

According to various embodiments, the cap 2 is a single-material unit that can be made of polyethylene terephthalate (PET), high-density polyethylene (HDPE) or polypropylene (PP), or any other thermoplastic polymer. This type of cap 2 comprises shadow zones 21, i.e., surfaces that cannot be reached directly by an incident particle beam, by way of examples the zones below the projecting parts of the body 18, the skirt 27 and the roof 17 of the cap 2 according to the direction of movement of the particles.

The pulsed electron beam at the exit of the electron gun undergoes a diffusion in the direction of the caps 2 that pass (or are positioned step by step) in front of the electron bombardment window 8. The diffusion of the electrons is conditioned by the propagation environment. Thus, in one embodiment, when the sterilization chamber 4 is created under a vacuum-type external environment 11, the electrons that come from the electron gun constitute a beam that is diffused in a rectilinear manner and reach directly via the opening the surfaces of the cap 2 with a complex shape, first sterilizing the inside exposed surfaces that are reached by, for example, the roof 17 of the cap or the inside surfaces of its body 18.

In the embodiment shown in FIG. 3, the propagation of the electrons is considered in a gaseous external environment 11 (in particular air) that is preferably sterile. In a gaseous environment, a portion of electrons that come from the electron gun diffuse directly in the direction of the exposed surfaces of the cap 2, while another portion of electrons from this beam undergo phenomena of back scatter in the air. These phenomena of back scatter are due to collisions between the electrons and the particles of the gaseous external diffusion environment 11, for example elastic interactions that create deflections, i.e., modifications of angles of diffusion of the electrons without losses (or minimal losses) of energy. The arrow 22 of FIG. 4 represents, by way of example, the trajectory of an electron that undergoes, on two occasions, an elastic diffusion in the external environment 11 of gaseous propagation, i.e., modifications of directions of propagations without losses of kinetic energy. The electrons that come from the electron bombardment window 8, diffused in a rectilinear manner or deflected into the gaseous external environment 11, then impact certain specific zones of the cap 2 based on their trajectories, with these zones relating to exposed surfaces of the cap 2. These electrons are referred to below as primary electrons.

Advantageously, the primary electron beam is homogeneous enough to impact all of the exposed surfaces of the cap 2.

Based on the trajectories of the primary electrons, different physical phenomena are then observed:
A portion of the primary electrons penetrate into the material of the cap 2 and are diffused until they are absorbed. An increase in the dose of electrons in the material is then observed until a maximum penetration thickness is reached, based on the density of material of the cap 2 and the energy of the electrons. Here, dose is defined as the quantity of energy that comes from the electrons and that is absorbed by the material. This energy absorption results in particular from a transfer of energy from the electrons to the atoms of the material via inelastic collisions. Furthermore, the distribution of the electron dose of electrons is not gradual in the thickness of the material: this distribution depends on the penetration of electrons into the material. The penetration of electrons into the material is all the more important the higher the energy of the electrons and/or the lower the density of the material of the irradiated object;
A portion of the primary electrons is directly reflected on the surface of the cap 2, resulting from elastic or inelastic collisions with constituent particles of the material of the cap 2. Currently, this physical phenomenon is referred to under the term of electron back-scattering, also known under the English term “back-scattering.” By way of example, the left inset of FIG. 4 illustrates by an enlarged view the possible different trajectories 23, 24, 25, 28 of a back-scattered electron on the surface of the cap 2. The back-scattered electron can itself be diffused in a direct manner (rectilinear trajectory without deviation), such as the trajectory 24, or can again undergo one or more elastic collisions in the external environment 11 of gaseous propagation, such as for the trajectories 23, 25, 28. The trajectory 28 makes it possible in particular to reach and therefore to decontaminate a shadow zone that is located under the skirt 27;
Certain electrons penetrate into the material, are diffused in the former, and then undergo one or more elastic collisions before emerging therefrom. This physical phenomenon also relates to a situation of back-scattering of primary electrons. The number of reflections, therefore interactions of the interactions with the atoms of the material of the cap 2, as well as the probability of emerging therefrom, will be all the greater the higher the kinetic energy, and therefore the speed of the electrons. In particular, the elastic collisions of the primary electrons in the material are exposed to very small losses of energy of the latter, increasing their probability of back-scattering. In contrast, a series of inelastic collisions quickly leads to a loss of kinetic energy of the electrons and consequently their absorption by the material. By way of illustrative example, the right inset shows an enlargement of the trajectory 26 of an incident primary electron on the cap 2. This electron initially has a non-deflected trajectory between the electron emission window 8 and an exposed surface of the cap 2, penetrates, and then is diffused in the material of the cap 2, and then successively undergoes two reflections finally leading to its back-scattering in the gaseous environment. According to an embodiment, the pulsed electron beam at the exit of the electron gun also makes it possible to decontaminate necks of containers that pass (for example via a conveyor) or that are positioned step by step in front of the electron bombardment window 8. These necks can, for example, be an integral part of a preform, a bottle, a tube or else glued onto a packaging carton. According to various embodiments, the neck is a single-material unit that can be made of polyethylene terephthalate (PET), high-density polyethylene (HDPE) or polypropylene (PP) or any other thermoplastic polymer.

FIG. 5 illustrates a sample embodiment of decontamination of a container neck 30. This figure shows a cutaway view of a circular container that comprises a shoulder 29 and a neck 30 placed upstream. The opening of the neck 30 is turned toward the electron bombardment window 8. Advantageously, different trajectories of electrons that come from the pulsed electron beam at the outlet of the electron bombardment window 8 (not shown) make possible the decontamination of specific zones of the neck 30 of the container.

The container neck 30 that is illustrated has a complex shape and comprises the following elements:
An outside collar 31;
An outside transfer ring 32;
Outside threads 33;
An opening or rim 34;
An inside surface 35, here a flat surface.

The collar 31, the transfer ring 32, and the threads 33 all form projecting ribs (helicoidal in the case of the threads 33), although with various radial extensions.

This type of neck 30 also comprises shadow zones 21, i.e., surfaces that cannot be reached directly by an incident particle beam, by way of examples the zones below the collar 31, the transfer ring 32, and threads 33. The rim 34 and the inside surface 35 are exposed to exposed zones of the neck 30, i.e., zones that can be directly reached by a primary electron beam that comes from the electron bombardment window 8.

Just as in the case of the decontamination of the caps, the following physical phenomena are observed:
A portion of the primary electrons penetrate into the material of the neck 30 and are diffused until they are absorbed. The exposed zones of the neck 30, for example its rim 34 and its inside surface 35, are then decontaminated;
A portion of the primary electrons are directly reflected onto the different surfaces of the neck 30 and/or the container, resulting from elastic or inelastic collisions with constituent particles of the material of the cap 30 and/or of the container. The trajectories 36, 37, 38, 39 illustrate examples of electron trajectories that are back-scattered into the air and that undergo elastic collisions on the neck 30 or the container. It is noted that, for example, the trajectory 39 makes it possible to reach the shadow zone 21 below the collar 31 via an elastic collision on the shoulder 29 of the container, then followed by a back-scattering, resulting from a collision of the electrons with particles from the propagation environment; a portion of the electrons penetrate into the material, are diffused in the former, and then undergo one or more elastic collisions before emerging therefrom. This situation is not illustrated here, but remains similar to the one that is described for the right inset of FIG. 4.

Thus, the primary electrons make it possible to decontaminate the exposed parts of the neck 30, while the shadow zones 21 are decontaminated using back-scattered electrons.

Advantageously, the back-scattered electrons make it possible to reach the shadow zone of the cap 2 and/or the neck 30 by their trajectories, and have high enough energy to be absorbed by the material of these zones, thus making possible their decontamination. Actually, the use of a pulsed electron flow makes it possible at the same time to obtain a high-intensity flow of electrons, ensuring the deposition of a sufficient lethal dose in the shadow zones, without thereby degrading the exposed surfaces that are exposed to the primary electron beam: the time of exposure of the cap 2 and/or the neck 30 to the electron bombardment is actually reduced to the minimum that is possible. In addition, it is advisable to note that the more heavy atoms a material comprises, the more electrons will be back-scattered by this material. A decontamination of caps and/or necks of containers with complex shapes by back-scattering of electrons is therefore particularly advantageous for caps and/or necks of containers made of the following materials: PET, HDPE, or PP.

One example of a set of parameters relative to the electron gun making it possible to obtain a pulsed electron flow and a back-scattering of electrons that can decontaminate caps 2 and/or necks of containers of complex shapes is provided below. So as to illustrate the advantages of the embodiments described above, these parameters are compared in relation to a configuration that relates to the current state of the art, using a continuous electron flow for the decontamination. The state of the art being considered is here an electron gun with scanning that uses a continuous electron beam for decontaminating caps. The assumption here is that the total treatment time for decontaminating a cap with such a gun is 1 second so as to provide a sufficient lethal dose of electrons and to cover all of the shadow zones. A potential difference of 250 kV is applied to the terminals of a filament diode of this gun, making it possible to obtain an anode current of 50 mA. By way of example, a continuous flow of electrons irradiating a cap for a period of 1 ms so as to calculate the electron dose received by the cap during this interval is considered.

Regarding the embodiments of the gun with pulsed electron flow of this application, the configurable parameters of this gun are the following: the number of pulses, the pulse time of a pulse, the discharge voltage that is applied to the terminals of the diode, the current of the anode of the diode, and the frequency of the emissions of the pulses. In this example, 10 pulses of 10 ns, generated at a frequency of 100 Hz, are used by applying a potential difference of 250 kV to the terminals of the diode with an anode current of 5 kA. Furthermore, the recharging time of the electron gun before being able to generate a new pulse is approximately 10 ms here.

Finally, a cap of a mass of 3 g and comprising a back-scattering coefficient of 0.07% will be assumed. The results that are obtained are summarized in the table below.

| | Sample | State of the Art: |
|---|---|---|
| | Embodiment: | Continuous |
| | Pulsed Electron | Electron |
| Parameters | Flow | Flow |
| N: Number of Pulses | 10 | 1 |
| Tpulse: Pulse Time (Unit: | 10 | 1,000,000 |
| ns, nanoseconds) | | |
| I: Anode Discharge | 5 | 0.00005 |
| Current (Unit: kA, | | |
| kiloampere) | | |
| U: Discharge Voltage | 250 | 250 |
| (Unit: kV, kilovolt) | | |
| m: Cap Mass | 3 | 3 |
| (Unit: g, gram) | | |
| Texpo: Time of Total | 0.0001 | 1 |
| Exposure of the Cap to | | |
| the Electron Flow (Unit: | | |
| ms, millisecond); Texpo = | | |
| N*Tpulse | | |
| Tcharge: Charge Time of | 9.99999 | 0 |
| the Electron Gun (Unit: | | |
| ms, millisecond) | | |
| T-Treatment: Total | 100 | 1,000 |
| Treatment Time of a Cap | | |
| (Unit: ms, millisecond); | | |
| T-Treatment = (Tpulse + | | |
| Tcharge)*N | | |
| Nmax: Maximum | 36,000 | 3,600 |
| Number of Caps Treated | | |
| per Hour: Nmax = | | |
| 3,600/T-Treatment | | |
| E: Transmitted Energy | 125 | 12.5 |
| (Unit: Joule, J) | | |
| E = N*U*I*T | | |
| D: Dose Received (Unit: | 41.66666667 | 4.166666667 |
| kilogray, kGy) | | |
| D = E/M | | |
| Qpulse: Quantity of | 5.00E−05 | 5.00E−05 |
| Electricity per Pulse | | |
| (Unit: Coulomb, C); | | |
| Qpulse = I*Tpulse | | |
| Qtot: Total Quantity of | 5.00E−04 | 5.00E−05 |
| Electricity (Unit: | | |
| Coulomb, C); Qtot = | | |
| Qpulse*N | | |
| η: Back-Scattering | 0.07 | 0.07 |
| Coefficient of the Material | | |
| of the Cap (%) | | |
| Qretro: Quantity of | 3.50E−05 | 3.50E−06 |
| Back-Scattered Electricity | | |
| (Unit: Coulomb, C); | | |
| Qretro = η*Qtot | | |

The example provided above illustrates several advantages that result from using a pulsed electron gun. In particular, the use of an anode current with a much higher value than the one used in the state of the art makes possible very short irradiation times while making possible the distribution of a much higher electron dose, here ten times more than in the state of the art. Thus, the quantity of electricity associated with back-scattered electrons is also higher and makes it possible to decontaminate correctly the shadow zones of the cap. In contrast, with the electron doses received in the state of the art being smaller, the same holds true for the quantity of energy of back-scattered electrons, which greatly limits the covering of shadow zones. In addition, it is observed that the use of a pulsed electron flow makes possible much shorter treatment times and therefore the decontamination of a much higher cap number during the same time period.

Experimental works for the purpose of decontaminating caps and/or necks of containers with complex shapes have led to identifying values of electron doses making possible an effective treatment of these caps and/or necks of containers. Preferably, the values of these doses are in a range of between 15 and 50 kGy.

Thus, according to various embodiments, other combinations of parameters can be selected in addition to the preceding example, making it possible to obtain electron doses located in this range. The table below specifies the range of these parameters:

| Parameters | Broad Range | Limited Range | Example |
|---|---|---|---|
| N: Number of | 5 to 200 | 10 to 100 | 10 |
| Pulses | | | |
| Tpulse: Pulse | 5 to 250 | 10 to 125 | 15 |
| Time (unit: ns, | | | |
| nanoseconds) | | | |
| I: Anode | 1 to 20 | 2 to 10 | 3.5 |
| Discharge Current | | | |
| (Unit: kA, | | | |
| kiloampere) | | | |
| U: Discharge | 75 to 500 | 200 to 300 | 250 |
| Voltage (Unit: kV, | | | |
| kilovolt) | | | |
| f: Frequency of | 50-500 | 100 to 200 | 100 |
| Pulses (Unit: Hz, | | | |
| Hertz) | | | |

In addition, according to various embodiments, so as to be able also to reduce the decontamination time of the caps and/or necks of containers, a number of pulsed electron guns can be used simultaneously. Since the parallel use of several guns is known to one skilled in the art, this embodiment makes it possible in particular to be able also to reduce the application time of a pulse on the object that is to be treated.

Advantageously, the above-described embodiments make it possible to provide a method for decontamination of caps and/or necks of containers that is efficient (reduction

1. Method for decontaminating caps (2) or necks (30) of containers by electron bombardment, each cap (2) comprising a roof (17), a body (18) projecting from a peripheral edge of the roof (17), this body (18) having an opening opposite the roof (17), ribs (19, 20) projecting from an inside face of the body (18) and/or an inside face of the roof (17), each neck (30) comprising ribs (33) and an opening (34), the ribs (19, 20, 33) having shadow zones (21), with this method comprising:
an operation for passage or positioning of the caps (2) or necks (30) in front of an electron bombardment window (8), with the opening of the caps (2) or necks (30) being turned toward this window (8);
an electron bombardment operation of caps (2) or necks (30), during the passage or positioning of the caps or necks (30) in front of the window (8);
wherein the bombardment is carried out by means of a pulsed electrical field that comprises a series of electric pulses of predetermined frequency, duration and intensity in such a way as to obtain primary electrons and back-scattered electrons, respectively making possible the decontamination of exposed zones and shadow zones of the caps (2) or necks (30).

2. Method according to claim 1, where the frequency is in a range of between 50 and 500 Hertz.

3. Method according to claim 1, where the frequency of electric pulses is 100 Hertz.

4. Method according to claim 1, where the electric pulse time is in a range of between 5 and 250 nanoseconds.

5. Method according to claim 1, where the electric pulse time is 10 nanoseconds.

6. Method according to claim 1, where the intensity of the electric pulses is between 1 and 20 kiloamperes.

7. Method according to claim 1, where the intensity of electric pulses is 5 kiloamperes.

8. System for decontaminating caps (2) or necks (30) of containers by electron bombardment, each cap (2) comprising a roof (17), a body (18) projecting from a peripheral edge of the roof (17), with this body (18) having an opening opposite to the roof (17), ribs (19, 20) projecting from an inside face of the body (18) and/or an inside face of the roof (17), each neck (30) comprising ribs (33) and an opening (34), with the ribs (19, 20, 33) having shadow zones (21), this system comprising:
means for passage or positioning of caps (2) or necks (30) of containers in front of an electron bombardment window (8), with the opening of the caps (2) or necks (30) of containers being turned toward this window (8);
means for electron bombardment of caps (2) or necks (30) of containers, during the passage or positioning of the caps (2) or necks (30) of containers in front of the window (8),
wherein the electron bombardment means are arranged to generate a pulsed electrical field that comprises a series of electric pulses of predetermined frequency, duration and intensity in such a way as to obtain primary electrons and back-scattered electrons, respectively making possible the decontamination of exposed zones and shadow zones of the caps (2) or necks (30).

9. System according to claim 8, where the caps (2) are adjacent to one another and pass along a predetermined transport path and at a predetermined speed, using a preestablished transport device (3).

10. System according to claim 9, where the transport device (3) is created by a set of rails.

11. Method according to claim 2, where the frequency of electric pulses is 100 Hertz.

12. Method according to claim 2, where the electric pulse time is in a

13. Method according to claim 2, where the intensity of the electric pulses is between 1 and 20 kiloamperes.
